(19) United States
(12) Patent Application Publication
(10) Pub. No.: US 2023/0304098 A1
(43) Pub. Date: Sep. 28, 2023

(54) METHODS AND SYSTEMS FOR CELL-FREE DNA FRAGMENT SIZE DENSITIES TO ASSESS CANCER

(71) Applicant: Delfi Diagnostics, Inc., Baltimore, MD (US)

(72) Inventors: Jacob Carey, Baltimore, MD (US); Nicholas C. Dracopoli, Baltimore, MD (US); Sian Jones, Baltimore, MD (US)

(21) Appl. No.: 18/019,448

(22) PCT Filed: Aug. 17, 2021
(86) PCT No.: PCT/US2021/046272

§ 371 Date: Feb. 2, 2023

Related U.S. Application Data
(60) Provisional application No. 63/163,434, filed on Mar. 19, 2021.
(60) Provisional application No. 63/067,244, filed on Aug. 18, 2020.

(51) Int. Cl. C12Q 1/6886; C12Q 1/6869; G16B 30/00; G16B 40/20

(52) U.S. Cl. CPC C12Q 1/6886; C12Q 1/6869; G16B 30/00; G16B 40/20; C12Q 2600/166; C12Q 2600/156

(57) ABSTRACT

The present disclosure provides methods and systems that utilize analysis of cell-free DNA (cfDNA) fragment size density in a sample obtained from a patient to diagnose and predict cancer status. Also provided is a system including a sequencer configured to generate a low-coverage whole genome sequencing data set for a sample.

### CROSS-REFERENCE TO RELATED APPLICATIONS

This application claims benefit of priority under 35 U.S.C. § 119(e) of U.S. Provisional Application Nos. 63/067,244 filed Aug. 18, 2020 and 63/163,434 filed Mar. 19, 2021. The disclosures of the prior applications are considered part of and are herein incorporated by reference in the disclosure of this application in their entirety.

### BACKGROUND OF THE INVENTION

### Field of the Invention

The invention relates generally to genetic analysis and more specifically to methods and systems for analysis of cell-free DNA fragment size densities to detect and/or assess cancer in a subject.

### Background Information

Much of the morbidity and mortality of human cancers world-wide is a result of the late diagnosis of these diseases, where treatments are less effective. Unfortunately, clinically proven biomarkers that can be used to broadly diagnose and treat patients with early cancer are not widely available.

Analyses of cell-free DNA (cfDNA) suggests that such approaches may provide new avenues for early diagnosis. Circulating tumor DNA (ctDNA) fragments have been shown to be on average shorter than other cfDNA from non-tumor cells. Previous work has explored separating fragments into groups of different sizes caused by binding to histone core or linker proteins (e.g., short and long, or mutually exclusive sets of sizes) and using counts of these fragments to quantify ctDNA and/or classify individual samples as having presence/absence of tumor. However, previous studies ignore the importance of the shape of the curve of fragment size density.

As such, a cancer detection and/or assessment method that utilizes analysis of the shape of the curve of fragment size density is desirable to allow for more robust and reliable detection of cancer in a subject.

### SUMMARY OF THE INVENTION

The present disclosure provides methods and systems that utilize analysis of a shape of a curve of cfDNA fragment size density in a sample obtained from a patient. The shape of the curve is demonstrated herein to be strongly predictive of cancer status.

As such, in one embodiment, the present invention provides a method of determining the cancer status of a subject. The method includes: (a) analyzing a shape of a curve of cfDNA fragment size density in a sample from a subject, wherein a difference in the shape of the curve of the cfDNA fragment size density from the subject and that from a reference sample of a healthy subject, is indicative of cancer in the subject; and (b) optionally administering a cancer treatment to the subject.

In another embodiment, the invention provides a method of determining DNA-nucleosome interaction dynamics in a subject which includes analyzing a shape of a curve of cfDNA fragment size density in a subject using the method of the invention. In certain aspects, the shape of the curve is indicative of the DNA-nucleosome interaction dynamics.

In yet another embodiment, the invention provides a method of predicting cancer status of a subject. The method includes: (a) analyzing a shape of a curve of cfDNA fragment size density in a sample obtained from the subject; (b) comparing the shape of the curve of cfDNA fragment size density of the sample to a reference curve shape; and (c) detecting cancer in the subject when the shape of the curve of cfDNA fragment size density of the sample is different from the reference curve shape, thereby predicting the cancer status of the subject.

In another embodiment, the invention provides a method of diagnosing and treating cancer in a subject. The method includes: (a) detecting cancer in the subject, wherein said detecting cancer includes analyzing a shape of a curve of cfDNA fragment size density in a sample obtained from the subject, comparing the shape of the curve of cfDNA fragment size density of the sample to a reference curve shape, and detecting cancer in the subject when the shape of the curve of cfDNA fragment size density of the sample is different from the reference curve shape; and (b) administering to the subject a cancer treatment, thereby treating cancer in the subject.

In still another embodiment, the invention provides a method of monitoring cancer in a subject. The method includes: (a) determining cancer status in the subject, wherein the cancer status is determined by analyzing a shape of a curve of cfDNA fragment size density in a first sample obtained from the subject; comparing the shape of the curve of cfDNA fragment size density of the first sample to a reference curve shape; and detecting cancer in the subject when the shape of the curve of cfDNA fragment size density of the first sample is different from the reference curve shape; (b) administering a cancer treatment to the subject; (c) determining a shape of a curve of cfDNA fragment size density of a second sample obtained from the subject; and (d) comparing the shape of the curve of cfDNA fragment size density of the second sample to the shape of the curve of cfDNA fragment size density of the first sample and/or to the reference curve shape, thereby monitoring cancer in the subject.

In another embodiment, the invention provides a system for genetic analysis and assessing cancer. The system includes: (a) a sequencer configured to generate a low-coverage whole genome sequencing data set for a sample; and (b) a computer system. In various aspects, the computer system has a non-transitory computer readable medium with instructions to perform one or more of the following: (i) process the low-coverage whole genome sequencing data set to produce a curve of fragment size density of the sample; (ii) fit the curve of fragment size density of the sample to at least two different sets of established statistical parameters to produce at least two suggested curve fits; (iii) display the at least two suggested curve fits, enabling a user to select at least one of said at least two suggested curve fits for further processing; and (iv) display a suggested curve fit line corresponding to a selected suggested curve fit of (iii) together with a reference curve fit line, enabling a comparison between the selected suggested curve fit line and the reference curve fit line.

In various aspects of the invention analyzing the shape of the curve of cfDNA fragment size density includes analysis of various fragment sizes. In some aspects, analyzing the shape of the curve of cfDNA fragment size density excludes fragment sizes less than about 10, 50, 100 or 105 bp and greater than about 220, 250, 300, 350 bp or greater. In some aspects, analyzing the shape of the curve of cfDNA fragment size density excludes fragment sizes less than about 105 bp and greater than about 170 bp. In some aspects, analyzing the shape of the curve of cfDNA fragment size density is of dinucleosomal DNA fragments. In some aspects, analyzing the shape of the curve of cfDNA fragment size density excludes fragment sizes less than about 230, 240, 250, 260 bp and greater than about 420, 430, 440, 450 bp or greater. In some aspects, analyzing the shape of the curve of cfDNA fragment size density excludes fragment sizes less than about 260 bp and greater than about 440 bp.

In still another embodiment, the invention provides a non-transitory computer readable storage medium encoded with a computer program. The computer program includes instructions that when executed by one or more processors cause the one or more processors to perform operations to perform a method of the invention.

In yet another embodiment, the invention provides a computing system. The system includes a memory, and one or more processors coupled to the memory, with the one or more processors being configured to perform operations that implement a method of the invention.

In yet another embodiment, the invention provides a system for genetic analysis and assessing cancer that includes: (a) a sequencer configured to generate a whole genome sequencing data set for a sample; and (b) a non-transitory computer readable storage medium and/or a computer system of the invention.

### BRIEF DESCRIPTION OF THE FIGURES

FIG. 1 is a graphical plot showing data generated using the methodology of the disclosure in one embodiment of the invention.

FIG. 2 is a graphical plot showing data generated using the methodology of the disclosure in one embodiment of the invention.

FIG. 3 is a graphical plot showing data generated using the methodology of the disclosure in one embodiment of the invention.

FIG. 4 is a graphical plot showing data generated using the methodology of the disclosure in one embodiment of the invention.

FIG. 5 is a graphical plot showing data generated using the methodology of the disclosure in one embodiment of the invention.

FIG. 6 is an image showing the various states produced by RSC sliding of dinucleosomes. Montel et al., (2011. “RSC remodeling of oligo-nucleosomes: an atomic force microscopy study.” Nucleic Acids Research 39 (7): 2571-2579.)

FIG. 7 is an image showing examples of potential endonuclease cleavage sites resulting in varying fragment sizes. Only two sites are shown per state, but cleavage may occur at multiple different positions in linker DNA. Montel et al., (2011).

FIG. 8 is a graphical plot showing data generated using the methodology of the disclosure in one embodiment of the invention.

FIG. 9 is a graphical plot showing data generated using the methodology of the disclosure in one embodiment of the invention.

### DETAILED DESCRIPTION OF THE INVENTION

The present invention is based on innovative methods and systems which utilize analysis of the shape of the curve of cfDNA fragment size density from cfDNA in a patient derived sample. As discussed herein, the present invention quantifies the shape of the curve in two approaches using: 1) polynomial regression; and 2) Bayesian finite mixture models. The methods and systems of the invention represent a novel approach for summarizing DNA-nucleosome interaction dynamics as they relate to cancer status of a subject.

Before the present compositions and methods are described, it is to be understood that this invention is not limited to the particular methods and systems described, as such methods and systems may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present disclosure provides innovative methods and systems for analysis cfDNA fragment size density to detect or otherwise assess cancer.

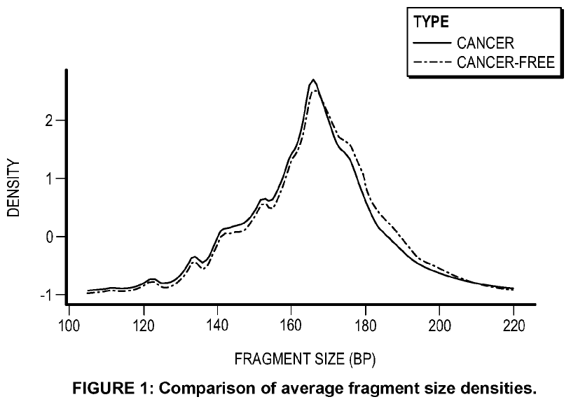

In one aspect, the data used to develop the methodology of the invention for quantifying the shape of the curve of the fragment size density is based on shallow whole genome sequence data (1-2x coverage). FIG. 1 shows a comparison of average fragment size densities. In FIG. 1, the average fragment size density by cancer and cancer-free individuals, normalized by amount of DNA reads is plotted.

As indicated in prior studies on average, cancer-free individuals have longer cfDNA fragments (average size of 167.09 bp) whereas individuals with cancer have shorter cfDNA fragments (average size of 164.88 bp). However, FIG. 1 shows differences in the overall shape of these densities beyond an increase of short fragments. Furthermore, these differences are not captured by periodicity of specific peaks, such as that utilized in prior efforts. Instead, these differences in fragment size densities may represent differences in DNA-nucleosome interaction dynamics and require alternative approaches beyond counts of bins or periodicity to accurately model. The methods described herein represent a novel approach for summarizing DNA-nucleosome interaction dynamics as they relate to cancer status of a plasma donor.

In various aspects, the present disclosure demonstrates that the fragment size density may be modeled as a mixture of distributions, the parameters of which may be predictive of cancer status. In some aspects, the present disclosure illustrates use of fragment sizes that closely correspond to the size of a single nucleosome (less than 260 bp in size). These typically include a histone octomer wrapped with 147 bp of DNA), together with an H1 histone and linker DNA (20 bp), giving an observed 167 bp size major peak. The disclosure also illustrates use of cfDNA fragments greater than 260 bp likely including two nucleosomes which result in a peak with a median of 334 bp.

Accordingly, in one embodiment, the invention provides a method of determining the cancer status of a subject. The method includes: (a) analyzing a shape of a curve of cfDNA fragment size density in a sample from a subject, wherein a difference in the shape of the curve of the cfDNA fragment size density from the subject and that from a reference sample of a healthy subject, is indicative of cancer in the subject; and (b) optionally administering a cancer treatment to the subject.

In another embodiment, the invention provides a method of predicting cancer status of a subject. The method includes: (a) analyzing a shape of a curve of cfDNA fragment size density in a sample obtained from the subject; (b) comparing the shape of the curve of cfDNA fragment size density of the sample to a reference curve shape; and (c) detecting cancer in the subject when the shape of the curve of cfDNA fragment size density of the sample is different from the reference curve shape, thereby predicting the cancer status of the subject.

In still another embodiment, the invention provides a method of treating cancer in a subject. The method includes: (a) detecting cancer in the subject, wherein said detecting cancer includes analyzing a shape of a curve of cfDNA fragment size density in a sample obtained from the subject, comparing the shape of the curve of cfDNA fragment size density of the sample to a reference curve shape, and detecting cancer in the subject when the shape of the curve of cfDNA fragment size density of the sample is different from the reference curve shape; and (b) administering to the subject a cancer treatment, thereby treating cancer in the subject.

In another embodiment, the invention provides a method of monitoring cancer in a subject. The method includes: (a) determining cancer status in the subject, wherein the cancer status is determined by analyzing a shape of a curve of cfDNA fragment size density in a first sample obtained from the subject; comparing the shape of the curve of cfDNA fragment size density of the first sample to a reference curve shape; and detecting cancer in the subject when the shape of the curve of cfDNA fragment size density of the first sample is different from the reference curve shape; (b) administering a cancer treatment to the subject; (c) determining a shape of a curve of cfDNA fragment size density of a second sample obtained from the subject; and (d) comparing the shape of the curve of cfDNA fragment size density of the second sample to the shape of the curve of cfDNA fragment size density of the first sample and/or to the reference curve shape, thereby monitoring cancer in the subject.

In various aspects, the methods of the invention include analyzing the shape of the curve of cfDNA fragment size density by fitting a finite mixture of distributions to counts of fragment sizes. In certain aspects, fitting the finite mixture of distributions to counts of fragment sizes includes quantifying components of the sample which may include quantifying a plurality of curves of cfDNA fragment size density. In some aspects, the sample includes about 2, 3, 4, 5, 6, 7, 8, 9, 10, 11, 12, 13, 14, 15 or more components. In one illustrative aspect, as discussed in Example 1, the sample includes 12 components.

In various aspects, the distributions include truncated normal distributions.

In various aspects, the methods include characterizing the components of a sample by statistical parameters and contribution to the overall mixture. Such statistical parameters may include, but are not limited to mean, variance and/or shape, and weight.

The methods of the invention may further include assessing convergence by determining that the statistical parameters have a multivariate potential scale reduction factor less than or equal to or about 1.5, 1.4, 1.3, 1.2, 1.1 or 1.0

In various aspects, analyzing the shape of the curve of cfDNA fragment size density includes excluding successive ranges of fragment sizes less than about 10, 50, 100 or 105 bp and greater than about 220, 250, 300, 350 bp or more. In one aspect, analyzing the shape of the curve of cfDNA fragment size density comprises excluding fragment sizes less than 105 bp and greater than 170 bp.

In certain aspects, analyzing the shape of the curve of cfDNA fragment size density is of dinucleosomal DNA fragments. In some aspects, analyzing the shape of the curve of cfDNA fragment size density excludes fragment sizes less than about 230, 240, 250, 260 bp and greater than about 420, 430, 440, 450 bp or greater. In one aspect, analyzing the shape of the curve of cfDNA fragment size density excludes fragment sizes less than 260 bp and greater than 440 bp.

Additionally, analyzing the shape of the curve of cfDNA fragment size density comprises quantifying the shape of the curve using coefficients of a polynomial regression fit to counts of fragments of a given length. As shown in Example 1, the methods may further include standardizing the counts of fragments to have a mean of 0 and a variance of 1.

In various aspects, analyzing the shape of the curve of cfDNA fragment size density may include one or more of: (i) processing a sample from the subject comprising cfDNA fragments into sequencing libraries; (ii) subjecting the sequencing libraries to low-coverage whole genome sequencing to obtain sequenced fragments; (iii) mapping the sequenced fragments to a genome to obtain windows of mapped sequences; and (iv) analyzing the windows of mapped sequences to determine cfDNA fragment lengths.

In certain aspects, the mapped sequences include tens to thousands of genomic windows, such as 10, 50, 100 to 1,000, 5,000, 10,000 or more windows. Such windows may be non-overlapping or overlapping and include about 1, 2, 3, 4, 5, 6, 7, 8, 9 or 10 million base pairs.

In various aspects, a cfDNA fragmentation profile is determined within each window. As such, the invention provides methods for determining a cfDNA fragmentation profile in a subject (e.g., in a sample obtained from a subject). As used herein, the terms “fragmentation profile,” “position dependent differences in fragmentation patterns,” and “differences in fragment size and coverage in a position dependent manner across the genome” are equivalent and can be used interchangeably.

In some aspects, determining a cfDNA fragmentation profile in a subject can be used in identifying a subject as having cancer by analyzing a shape of a curve of cfDNA fragment size density. For example, cfDNA fragments obtained from a subject (e.g., from a sample obtained from a subject) can be subjected to low-coverage whole-genome sequencing, and the sequenced fragments can be mapped to the reference human genome (e.g., in non-overlapping windows) and assessed to determine a cfDNA fragmentation profile and the shape of the curve of cfDNA fragment size density analyzed. As described herein, a cfDNA fragmentation profile of a subject having cancer is more heterogeneous (e.g., in fragment lengths) than a cfDNA fragmentation profile of a healthy subject (e.g., a subject not having cancer).

In some aspects, the cfDNA fragmentation profile includes a fragment size of greatest frequency defining a peak of the curve of cfDNA fragment size density. In some aspects, the cfDNA fragmentation profile includes a fragment size distribution having fragment sizes of varying frequency. In some aspects, the cfDNA fragmentation profile includes a ratio of small cfDNA fragments to large cfDNA fragments in said windows of mapped sequences. In some aspects, the cfDNA fragmentation profile includes the sequence coverage of small cfDNA fragments in windows across the genome. In some aspects, the cfDNA fragmentation profile includes the sequence coverage of large cfDNA fragments in windows across the genome. In some aspects, the cfDNA fragmentation profile includes the sequence coverage of small and large cfDNA fragments in windows across the genome. In some aspects, the cfDNA fragmentation profile is over the whole genome. In some aspects, the cfDNA fragmentation profile is over a subgenomic interval.

A cfDNA fragmentation profile can include one or more cfDNA fragmentation patterns. A cfDNA fragmentation pattern can include any appropriate cfDNA fragmentation pattern. Examples of cfDNA fragmentation patterns include, without limitation, median fragment size, fragment size distribution, ratio of small cfDNA fragments to large cfDNA fragments, and the coverage of cfDNA fragments. In some aspects, a cfDNA fragmentation pattern includes two or more (e.g., two, three, or four) of median fragment size, fragment size distribution, ratio of small cfDNA fragments to large cfDNA fragments, and the coverage of cfDNA fragments. In some aspects, a cfDNA fragmentation profile can be a genome-wide cfDNA profile (e.g., a genome-wide cfDNA profile in windows across the genome). In some aspects, a cfDNA fragmentation profile can be a targeted region profile. A targeted region can be any appropriate portion of the genome (e.g., a chromosomal region). Examples of chromosomal regions for which a cfDNA fragmentation profile can be determined as described herein include, without limitation, a portion of a chromosome (e.g., a portion of 2q, 4p, 5p, 6q, 7p, 8q, 9q, 10q, 11q, 12q, and/or 14q) and a chromosomal arm (e.g., a chromosomal arm of 8q, 13q, 11q, and/or 3p). In some aspects, a cfDNA fragmentation profile can include two or more targeted region profiles.

In some aspects, a cfDNA fragmentation profile can be used to identify changes (e.g., alterations) in cfDNA fragment lengths. An alteration can be a genome-wide alteration or an alteration in one or more targeted regions/loci. A target region can be any region containing one or more cancer-specific alterations. In some aspects, a cfDNA fragmentation profile can be used to identify (e.g., simultaneously identify) from about 10 alterations to about 500 alterations (e.g., from about 25 to about 500, from about 50 to about 500, from about 100 to about 500, from about 200 to about 500, from about 300 to about 500, from about 10 to about 400, from about 10 to about 300, from about 10 to about 200, from about 10 to about 100, from about 10 to about 50, from about 20 to about 400, from about 30 to about 300, from about 40 to about 200, from about 50 to about 100, from about 20 to about 100, from about 25 to about 75, from about 50 to about 250, or from about 100 to about 200, alterations).

In various aspects, a cfDNA fragmentation profile can include a cfDNA fragment size pattern. cfDNA fragments can be any appropriate size. For example, in some aspects, a cfDNA fragment can be from about 50 base pairs (bp) to about 400 bp in length. As described herein, a subject having cancer can have a cfDNA fragment size pattern that contains a shorter median cfDNA fragment size than the median cfDNA fragment size in a healthy subject. A healthy subject (e.g., a subject not having cancer) can have cfDNA fragment sizes having a median cfDNA fragment size from about 166.6 bp to about 167.2 bp (e.g., about 166.9 bp). In some aspects, a subject having cancer can have cfDNA fragment sizes that are, on average, about 1.28 bp to about 2.49 bp (e.g., about 1.88 bp) shorter than cfDNA fragment sizes in a healthy subject. For example, a subject having cancer can have cfDNA fragment sizes having a median cfDNA fragment size of about 164.11 bp to about 165.92 bp (e.g., about 165.02 bp).

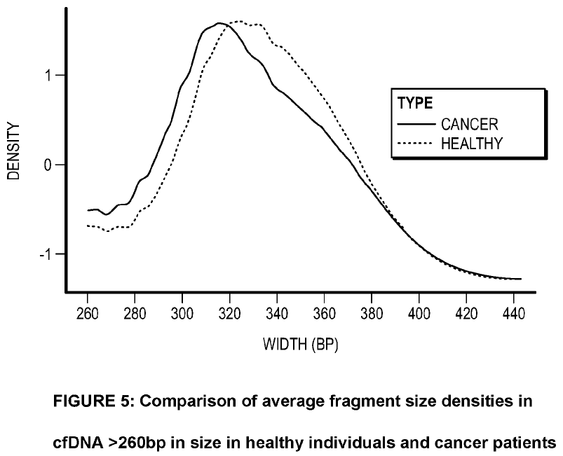

In some aspects, a dinucleosomal cfDNA fragment can be from about 230 base pairs (bp) to about 450 bp in length. As described herein, a subject having cancer can have a dinucleosomal cfDNA fragment size pattern that contains a shorter median dinucleosomal cfDNA fragment size than the median dinucleosomal cfDNA fragment size in a healthy subject. In some aspects as is seen in FIG. 5, it is evident that on average, cancer-free subjects have longer cfDNA fragments in the dinucleosomal range (average size of 334.75 bp) whereas subjects with cancer have shorter dinucleosomal cfDNA fragments (average size of 329.6 bp). As such, a healthy subject (e.g., a subject not having cancer) can have dinucleosomal cfDNA fragment sizes having a median cfDNA fragment size of about 334.75 bp. In some aspects, a subject having cancer can have dinucleosomal cfDNA fragment sizes that are shorter than dinucleosomal cfDNA fragment sizes in a healthy subject. For example, a subject having cancer can have dinucleosomal cfDNA fragment sizes having a median cfDNA fragment size of about 329.6 bp.

A cfDNA fragmentation profile can include a cfDNA fragment size distribution. As described herein, a subject having cancer can have a cfDNA size distribution that is more variable than a cfDNA fragment size distribution in a healthy subject. In some aspects, a size distribution can be within a targeted region. A healthy subject (e.g., a subject not having cancer) can have a targeted region cfDNA fragment size distribution of about 1 or less than about 1. In some aspects, a subject having cancer can have a targeted region cfDNA fragment size distribution that is longer (e.g., 10, 15, 20, 25, 30, 35, 40, 45, 50 or more bp longer, or any number of base pairs between these numbers) than a targeted region cfDNA fragment size distribution in a healthy subject. In some aspects, a subject having cancer can have a targeted region cfDNA fragment size distribution that is shorter (e.g., 10, 15, 20, 25, 30, 35, 40, 45, 50 or more bp shorter, or any number of base pairs between these numbers) than a targeted region cfDNA fragment size distribution in a healthy subject. In some aspects, a subject having cancer can have a targeted region cfDNA fragment size distribution that is about 47 bp smaller to about 30 bp longer than a targeted region cfDNA fragment size distribution in a healthy subject. In some aspects, a subject having cancer can have a targeted region cfDNA fragment size distribution of, on average, a 10, 11, 12, 13, 14, 15, 15, 17, 18, 19, 20 or more bp difference in lengths of cfDNA fragments. For example, a subject having cancer can have a targeted region cfDNA fragment size distribution of, on average, about a 13 bp difference in lengths of cfDNA fragments. In some aspects, a size distribution can be a genome-wide size distribution.

A cfDNA fragmentation profile can include a ratio of small cfDNA fragments to large cfDNA fragments and a correlation of fragment ratios to reference fragment ratios. As used herein, with respect to ratios of small cfDNA fragments to large cfDNA fragments, a small cfDNA fragment can be from about 100 bp in length to about 150 bp in length. As used herein, with respect to ratios of small cfDNA fragments to large cfDNA fragments, a large cfDNA fragment can be from about 151 bp in length to 220 bp in length. As described herein, a subject having cancer can have a correlation of fragment ratios (e.g., a correlation of cfDNA fragment ratios to reference DNA fragment ratios such as DNA fragment ratios from one or more healthy subjects) that is lower (e.g., 2-fold lower, 3-fold lower, 4-fold lower, 5-fold lower, 6-fold lower, 7-fold lower, 8-fold lower, 9-fold lower, 10-fold lower, or more) than in a healthy subject. A healthy subject (e.g., a subject not having cancer) can have a correlation of fragment ratios (e.g., a correlation of cfDNA fragment ratios to reference DNA fragment ratios such as DNA fragment ratios from one or more healthy subjects) of about 1 (e.g., about 0.96). In some aspects, a subject having cancer can have a correlation of fragment ratios (e.g., a correlation of cfDNA fragment ratios to reference DNA fragment ratios such as DNA fragment ratios from one or more healthy subjects) that is, on average, about 0.19 to about 0.30 (e.g., about 0.25) lower than a correlation of fragment ratios (e.g., a correlation of cfDNA fragment ratios to reference DNA fragment ratios such as DNA fragment ratios from one or more healthy subjects) in a healthy subject.

The presently described methods and systems are useful for detecting, predicting, treating and/or monitoring cancer status in a subject. Any appropriate subject, such as a mammal can be assessed, monitored, and/or treated as described herein. Examples of some mammals that can be assessed, monitored, and/or treated as described herein include, without limitation, humans, primates such as monkeys, dogs, cats, horses, cows, pigs, sheep, mice, and rats. For example, a human having, or suspected of having, cancer can be assessed using a method described herein and, optionally, can be treated with one or more cancer treatments as described herein.

A subject having, or suspected of having, any appropriate type of cancer can be assessed and/or treated (e.g., by administering one or more cancer treatments to the subject) using the methods and systems described herein. A cancer can be any stage cancer. In some aspects, a cancer can be an early stage cancer. In some aspects, a cancer can be an asymptomatic cancer. In some aspects, a cancer can be a residual disease and/or a recurrence (e.g., after surgical resection and/or after cancer therapy). A cancer can be any type of cancer. Examples of types of cancers that can be assessed, monitored, and/or treated as described herein include, without limitation, colorectal cancer, lung cancer, breast cancer, gastric cancers, pancreatic cancer, bile duct cancer, head and neck cancer, kidney cancer, bone cancer, brain cancer, hematopoietic cancer and ovarian cancer.

When treating a subject having, or suspected of having, cancer as described herein, the subject can be administered one or more cancer treatments. A cancer treatment can be any appropriate cancer treatment. One or more cancer treatments described herein can be administered to a subject at any appropriate frequency (e.g., once or multiple times over a period of time ranging from days to weeks). Examples of cancer treatments include, without limitation adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g., a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some aspects, a cancer treatment can reduce the severity of the cancer, reduce a symptom of the cancer, and/or to reduce the number of cancer cells present within the subject.

In some aspects, a cancer treatment can be a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art; see, e.g., the guidelines for therapy from the American Society of Clinical Oncology (ASCO), European Society for Medical Oncology (ESMO), or National Comprehensive Cancer Network (NCCN).

When monitoring a subject having, or suspected of having, cancer as described herein, the monitoring can be before, during, and/or after the course of a cancer treatment. Methods of monitoring provided herein can be used to determine the efficacy of one or more cancer treatments and/or to select a subject for increased monitoring. In some aspects, the monitoring can include analyzing a shape of a curve of cfDNA fragment size density in a sample obtained from a subject. For example, a shape of a curve of cfDNA fragment size density can be obtained before administering one or more cancer treatments to a subject having, or suspected or having, cancer, one or more cancer treatments can be administered to the subject, and one or more curves of cfDNA fragment size density can be obtained during the course of the cancer treatment. In some aspects, a shape of a curve of cfDNA fragment size density can change during the course of cancer treatment (e.g., any of the cancer treatments described herein). For example, a shape of a curve of cfDNA fragment size density indicative that the subject has cancer can change to a shape of a curve of cfDNA fragment size density indicative that the subject does not have cancer.

In some aspects, the monitoring can include conventional techniques capable of monitoring one or more cancer treatments (e.g., the efficacy of one or more cancer treatments). In some aspects, a subject selected for increased monitoring can be administered a diagnostic test (e.g., any of the diagnostic tests disclosed herein) at an increased frequency compared to a subject that has not been selected for increased monitoring. For example, a subject selected for increased monitoring can be administered a diagnostic test at a frequency of twice daily, daily, bi-weekly, weekly, bi-monthly, monthly, quarterly, semi-annually, annually, or any at frequency therein.

In various aspects, DNA is present in a biological sample taken from a subject and used in the methodology of the invention. The biological sample can be virtually any type of biological sample that includes DNA. The biological sample is typically a fluid, such as whole blood or a portion thereof with circulating cfDNA. In embodiments, the sample includes DNA from a tumor or a liquid biopsy, such as, but not limited to amniotic fluid, aqueous humor, vitreous humor, blood, whole blood, fractionated blood, plasma, serum, breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, prostatic fluid, nipple aspirate fluid, lachrymal fluid, perspiration, cheek swabs, cell lysate, gastrointestinal fluid, biopsy tissue and urine or other biological fluid. In one aspect, the sample includes DNA from a circulating tumor cell.

As disclosed above, the biological sample can be a blood sample. The blood sample can be obtained using methods known in the art, such as finger prick or phlebotomy. Suitably, the blood sample is approximately 0.1 to 20 ml, or alternatively approximately 1 to 15 ml with the volume of blood being approximately 10 ml. Smaller amounts may also be used, as well as circulating free DNA in blood. Microsampling and sampling by needle biopsy, catheter, excretion or production of bodily fluids containing DNA are also potential biological sample sources.

The methods and systems of the disclosure utilize nucleic acid sequence information, and can therefore include any method or sequencing device for performing nucleic acid sequencing including nucleic acid amplification, polymerase chain reaction (PCR), nanopore sequencing, 454 sequencing, insertion tagged sequencing. In some aspects, the methodology or systems of the disclosure utilize systems such as those provided by Illumina, Inc, (including but not limited to HiSeq™ X10, HiSeq™ 1000, HiSeq™ 2000, HiSeq™ 2500, Genome Analyzers™, MiSeq™ NextSeq, NovaSeq 6000 systems), Applied Biosystems Life Technologies (SOLiD™ System, Ion PGM™ Sequencer, ion Proton™ Sequencer) or Genapsys or BGI MGI and other systems. Nucleic acid analysis can also be carried out by systems provided by Oxford Nanopore Technologies (GridiON™, MiniON™) or Pacific Biosciences (Pacbio™ RS II or Sequel I or II).

The present invention includes systems for performing steps of the disclosed methods and is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions.

Accordingly, the invention further provides a system for detecting, analyzing and/or assessing cancer. In various aspects, the system includes: (a) a sequencer configured to generate a low-coverage whole genome sequencing data set for a sample; and (b) a computer system and/or processor with functionality to perform a method of the invention.

In certain aspects, the computer system has a non-transitory computer readable medium with instructions to perform one or more of the following: (i) process the low-coverage whole genome sequencing data set to produce a curve of fragment size density of the sample; (ii) fit the curve of fragment size density of the sample to at least two different sets of established statistical parameters to produce at least two suggested curve fits; (iii) display the at least two suggested curve fits, enabling a user to select at least one of said at least two suggested curve fits for further processing; and (iv) display a suggested curve fit line corresponding to a selected suggested curve fit of (iii) together with a reference curve fit line, enabling a comparison between the selected suggested curve fit line and the reference curve fit line.

In some aspects, the computer system has a non-transitory computer readable medium with instructions to determine, for one or more chromosomal arms of a genome, the number of fragments between about 260 bp and 440 bp in length and calculate the proportion of dinucleosomal fragments per arm. In some aspects, the computer system has a non-transitory computer readable medium with instructions to determine, for one or more chromosomal arms of a genome, the number of fragments between about 260 bp and 440 bp in length, calculate the proportion of dinucleosomal fragments per arm, and calculate the number of dinucleosomal fragments in each chromosomal arm. In some aspects, the computer system has a non-transitory computer readable medium with instructions to determine, for one or more chromosomal arms of a genome, the number of fragments between about 260 bp and 440 bp in length, calculate the proportion of dinucleosomal fragments per arm, calculate the number of dinucleosomal fragments in each chromosomal arm, and generate a curve of fragment size density.

In some aspects, the computer system further includes one or more additional modules. For example, the system may include one or more of: an extraction unit operable to select suitable components for curve fitting analysis, a curve fitting unit operable to perform a finite mixture of normal distributions fitting or perform a polynomial regression fitting by using user-defined equations, a curve fitting goodness analysis unit operable to provide indicators for fitting quality generated by the curve fitting unit, a curve fitting parameters characterization unit operable to classify curve fitting parameters as compared to reference values, a characterization database for storing fitting coefficients and their characterization, or any combination thereof.

In some aspects, the computer system further includes a visual display device. The visual display device may be operable to display a curve fit line, a reference curve fit line, and/or a comparison of both.

Methods for detection and analysis according to various aspects of the present invention may be implemented in any suitable manner, for example using a computer program operating on the computer system. As discussed herein, an exemplary system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, include any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing information may be implemented in a single device or implemented in a plurality of devices. The software may be accessible via a network such that storage and processing of information takes place remotely with respect to users. The system according to various aspects of the present invention and its various elements provide functions and operations to facilitate detection and/or analysis, such as data gathering, processing, analysis, reporting and/or diagnosis. For example, in the present aspect, the computer system executes the computer program, which may receive, store, search, analyze, and report information relating to the human genome or region thereof. The computer program may comprise multiple modules performing various functions or operations, such as a processing module for processing raw data and generating supplemental data and an analysis module for analyzing raw data and supplemental data to generate quantitative assessments of a disease status model and/or diagnosis information.

The procedures performed by the system may comprise any suitable processes to facilitate analysis and/or cancer diagnosis. In one embodiment, the system is configured to establish a disease status model and/or determine disease status in a patient. Determining or identifying disease status may include generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example 1

### Detection of Cancer

In this example, the methodology of the present disclosure was utilized to detect cancer. The following provides an in-depth discussion of the methodology and process used for cancer detection. Using whole genome data from 421 plasma samples collected from donors consisting of 207 individuals with cancer and 214 cancer-free individuals, it is demonstrated how the shape of the fragment size density is calculated in a manner that relates to nucleosomal repositioning. Additionally, it is shown how the results are used to predict cancer status both alone and in conjunction with other developed approaches.

The two methodologies used to summarize the shape of the fragment size density are described previously herein and in more detail as follows.

In the first method (polynomial regression method), the coefficients of a polynomial regression fit to counts of fragments of a given length, fit separately by sample, were used. Fragments less than 105 bp and greater than 170 bp in size were excluded due to low counts of fragments and less prominent modes not easily captured by the polynomial regression. In detail, the counts of fragments of size N were standardized by sample to have mean 0 and variance 1. As input to this regression were the orthogonal polynomials of degrees one to twelve of the fragment sizes. Thus, for each regression model, the input is a matrix of size 66×12 and the output is the scaled counts. Extracted from each polynomial regression model are the coefficients. The polynomial regression model explicitly captures the shape of the fragment size density and implicitly models the contributions of nucleosome sliding and DNA loops.

In the second method (Bayesian finite mixture models), a finite mixture of truncated normal distributions were fit to the counts of fragment sizes. Fragment sizes less than 105 bp and greater than 220 bp were excluded due to low counts of DNA fragments outside of that range. Seven of the components correspond to the modes can be seen on the ascending side of the distribution. Three of the components correspond to the components seen on the descending side. One component characterizes the overall base and makes up more than 50% of the mixture in 98.33% of the samples. The final component captures the skew seen in the descending side of the fragment size density. Each component has a truncation of 220 bp to prevent underestimation of the variance in the components with larger means. Each of these components is characterized by a mean, variance, and contribution to the overall mixture. Non-exchangeable, moderately informative priors were placed on the mixture means and variances and a weakly informative Dirichlet prior on the mixture proportions. This model is fit per sample using a No-U-Turn-Sampler (Hoffman, Matthew D., and Andrew Gelman. 2014. “The No-U-Turn Sampler: Adaptively Setting Path Lengths in Hamiltonian Monte Carlo.” J. Mach. Learn. Res. 15 (1): 1593-1623.) with 2,000 samples (1,000 warm-up samples). Convergence is assessed by checking that all parameters have a multivariate potential scale reduction factor (Gelman, Andrew, and Donald B. Rubin. 1992. “Inference from Iterative Simulation Using Multiple Sequences.” Statistical Science 7 (4): 457-72. https://doi.org/10.1214/ss/1177011136.) less than or equal to 1.1. Models may also be fit using Variational Inference.

Results

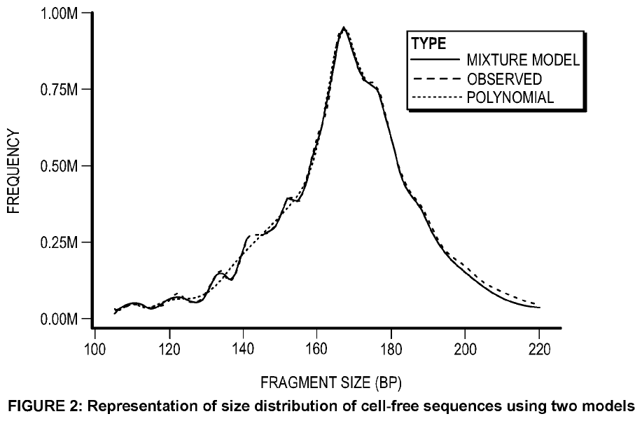

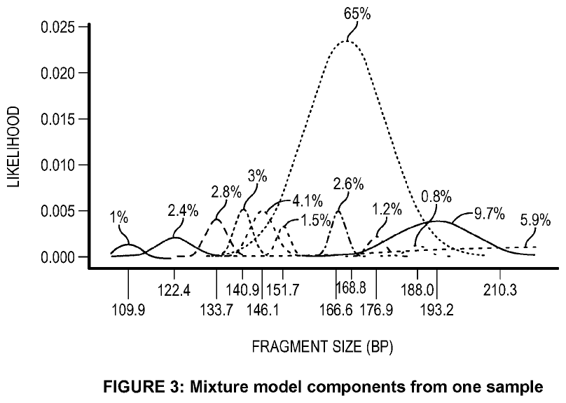

As seen in FIG. 2, while the polynomial model captures some of the nuances of the shape of the fragment size density, the mixture model provides a nearly exact fit to the shape of the fragment size density. Importantly, FIG. 3 shows the interpretability of the mixture model and makes the connection with underlying DNA-nucleosome interactions more explicit.

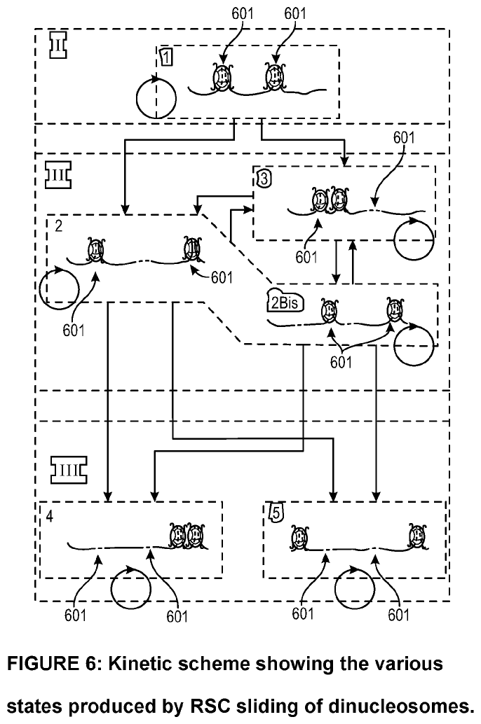

It is believed that the shape of fragment size densities may reflect DNA-nucleosome interaction dynamics. Lequieu et al., (Lequieu, Joshua, David C. Schwartz, and Juan J. de Pablo. 2017. “In Silico Evidence for Sequence-Dependent Nucleosome Sliding.” Proceedings of the National Academy of Sciences 114 (44): E9197—E9205. https://doi.org/10.1073/pnas.1705685114.) describe a variety of methods through which nucleosomes reposition within the genome. In particular, they develop a molecular model to describe DNA loops which are “introduced into one side of the nucleosome and then move along the histone core in an inchworm-like manner.” Lequieu et al., find that the location of these DNA loops are “insensitive to DNA sequence” and based on their molecular model, demonstrate in FIG. 6 a density plot of DNA loops by location on the histone. This density plot reflects many of the shapes of our densities shown in FIG. 2. Of note, both densities exhibit a similar pattern of small modes. The introduction of these loops may yield opportunities for endo-nuclease enzymes to cleave nucleosomal DNA inside of the linker histone. For example, a DNA loop at the end of the nucleosomal DNA might allow for the creation of a fragment that is slightly shorter than 167 bp. The farther along the histone the loop “inches,” the shorter the resulting cleaved fragment. Additionally, nucleosomes being repositioned by DNA loops may be cleaved at the linker histone and may appear as cfDNA fragments that are longer than 167 bp.

Based on the description in Lequieu et al., many of the components in the mixture model may be driven by DNA loop location and stability. With this understanding, periodicity and counts of fragment sizes will not capture these features of DNA loops. DNA loops may be more frequent at a certain location on the histone in a given sample. This will be reflected in the mixture proportion. Additionally, stability of the loops may be reflected variances of the components.

To evaluate the ability of the mixture model parameters to predict cancer status of the donors, the coefficients were used as features in a machine learning model in the same approach as described in Cristiano et al., (Cristiano, Stephen, Alessandro Leal, Jillian Phallen, Jacob Fiksel, Vilmos Adleff, Daniel C. Bruhm, Sarah Østrup Jensen, et al. 2019. “Genome-Wide Cell-Free DNA Fragmentation in Patients with Cancer.” Nature 570 (7761): 385-89. https://doi.org/10.1038/s41586-019-1272-6.). In short, a 10-fold, 10-repeat cross-validation of a Gradient Boosting Machine (Friedman, Jerome H. 2000. “Greedy Function Approximation: A Gradient Boosting Machine.” Annals of Statistics 29: 1189-1232.) with no hyper-parameter tuning was used. The following features were used.

1) Mixture model coefficients: the mixture model is fully described with 12 means, 12 variances, and 12 mixture proportions. For modeling, we exclude the 12th proportion, since it is a linear combination of the other 11 proportions. The 35 features were un-transformed.

2) Short/long ratios: similar to Cristiano et al., we calculated the number of GC-content corrected short (100-150 bp) and long (151-220 bp) fragments in 504 mutually exclusive 5 MB bins across the genome and divided the count of short by count of long as centered by sample.

3) Short/total coverage: as in the ratios, we used the coverage of short (100-150 bp) and total (100-220 bp) as features. These coverages are standardized to have mean 0 and standard deviation 1 by sample and type (short, total).

These three feature sets were used individually as inputs to the GBM as well as combinations of the mixture Model coefficients with (2) and (3). The results are reported in Table 1 as area under the ROC curve (AUC) and sensitivity at 95% and 98% specificity.

TABLE 1
Cross-validated results of cancer detection.

| Feature Set | AUC | Sens. (95% Spec.) | Sens. (98% Spec.) |
|---|---|---|---|
| Short/Long Ratio | 0.8902 | 0.5898 | 0.5146 |
| Short/Total Coverage | 0.9307 | 0.7670 | 0.6650 |
| Mixture | 0.9466 | 0.7330 | 0.6990 |
| Mixture,Ratio | 0.9547 | 0.7621 | 0.7039 |
| Mixture, Coverage | 0.9737 | 0.8301 | 0.7573 |

As illustrated in Table 1, the AUC of the mixture model coefficients indicates a strong predictor of cancer status. More indicative of utility for early cancer detection is sensitivity at a high specificity. While the mixture model coefficients have slightly lower sensitivity than the coverage parameters, combining the two shows both an improved AUC and sensitivity. The results of the combined features demonstrate how this novel summary of fragment size density can complement other genomic features to determine cancer status.

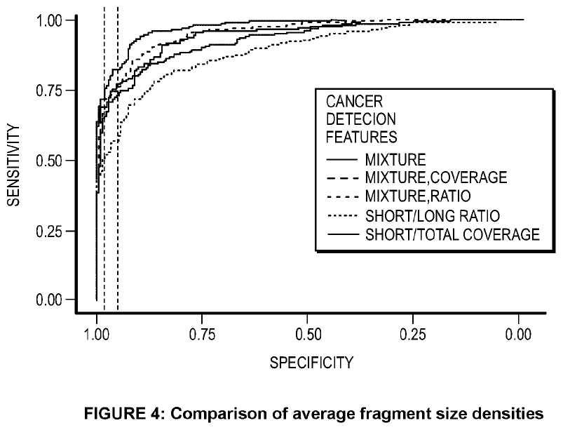

In FIG. 4 the ROC curves of these five models were plotted. These curves demonstrate the high sensitivity at even high specificities of the mixture coefficients as a feature as well the combination of mixture and coverage features. Reference lines indicate 95% and 98% specificity.

### Example 2

### Detection of Cancer Using Dinucleosomal Fragment Size Density

In this example, the methodology of the present disclosure was utilized to detect cancer. As discussed herein, ctDNA fragments have been shown to be on average shorter than other cfDNA from non-tumor cells. Previous work has explored separating fragments into groups of different sizes, e.g., short and long, or mutually exclusive sets of sizes and using counts of these bins to quantify ctDNA and/or classify individual samples as having presence/absence of a tumor.

As demonstrated herein, fragment size density may be modeled as a mixture of distributions, the parameters of which may be predictive of cancer status. These approaches all focus on fragment sizes that closely correspond to the size of a single nucleosome (less than 260 bp in size). These typically include a histone octomer wrapped with 147 bp of DNA), together with an H1 histone and linker DNA (20 bp), giving the observed 167 bp size major peak.

The study described in this Example focused on analyses of cfDNA fragments less than 260 bp from low coverage whole genome sequencing (1-2x coverage), likely including two nucleosomes which result in a peak with a median of 334 bp.

Methods

For each sample, the number of fragments is determined by base-pair between 260 bp and 440 bp for each chromosomal arm (excluding acrocentric chromosome arms). The proportion of dinucleosomal fragments per arm by width was calculated. These proportions across all 181 fragment sizes in a given arm sum to 1. This set of 181 proportions is defined as the dinucleosomal fragment size density. Each sample has 39 sets of dinucleosomal fragment size densities, calculated by non-acrocentric chromosome arm.

In a set of 48 purchased, exclusively cancer-free samples, these fragment size densities were calculated for each arm, and aggregated across all samples to represent 39 reference fragment size densities.

For each sample in the dataset, the similarity of the given sample/arm to the reference was determined by calculating the Kullback-Leibler divergence between the empirical sample/arm and the reference/arm. Additionally, the number of dinucleosomal fragments in each chromosome arm was calculated.

To evaluate the ability of these dinucleosomal parameters to predict cancer status of the donors, the inventors use the parameters as features in a machine learning model using the same approach as described in Cristiano et al. (Cristiano et al. 2019. “Genome-wide cell-free DNA fragmentation in patients with cancer.” Nature 570(7761): 385-389). In short, a 10-fold, 10-repeat cross-validation of a Gradient Boosting Machine (Friedman, Jerome H. 2000. “Greedy Function Approximation: A Gradient Boosting Machine.” Annals of Statistics 29: 1189-1232) is used with no hyper-parameter tuning. The following feature sets were used.

1) Dinucleosomal: The inventors calculated the Kullback-Leibler divergence between each sample/arm proportion of sizes and the reference. The number of dinucleosomal reads (260 bp-440 bp) per arm was also determined.

2) Short/total coverage: Similar to Cristiano et al. (2019) the inventors calculated the number of GC-content corrected short (100-150 bp) and total (100-220 bp) fragments in 504 mutually exclusive 5 MB bins across the genome. These coverages are standardized to have mean 0 and standard deviation 1 by sample and type (short, total).

These two feature sets were used individually as inputs to the GBM as well as the combination of the two features. The results are reported in Table 2 below, as area under the ROC curve (AUC) and sensitivity at 95% and 98% specificity.

Results

In FIG. 5, the average fragment size density is plotted, between 260 bp and 440 bp by cancer and cancer-free individuals, normalized by amount of DNA reads.

As is seen in FIG. 5, it is evident that on average, the cancer-free individuals have longer cfDNA fragments in the dinucleosomal range (average size of 334.75 bp) whereas the cancer patients have shorter cfDNA fragments (average size of 329.6 bp). Some studies have focused on mononucleosomal cfDNA and have shown that on average cfDNA fragments less than 260 bp in individuals with cancer are shorter than individuals without cancer. However, these efforts did not evaluate size distributions of dinucleosome cfDNA (less than 260 bp in size) in cancer patients or healthy individuals.

The inventors expect that the peak at 334 bp represents a dinucleosome, that is two adjacent nucleosomes with associated H1 and linker DNA, each encompassing 167 bp of DNA. Given that there is typically additional linker DNA between nucleosomes, repositioning of one or both nucleosomes would be required to support this hypothesis. One study demonstrated using atomic force microscopy (ATM) that nucleosomes can be repositioned in such a manner by the RSC (remodeling the structure of chromatin) complex as shown in FIG. 6. Of the five states identified, one of the most stable (#4) represents directly adjacent nucleosomes at the end of naked DNA and cleavage may occur at the 5′ and 3′ ends of this configuration. Remodeling is independent of DNA length.

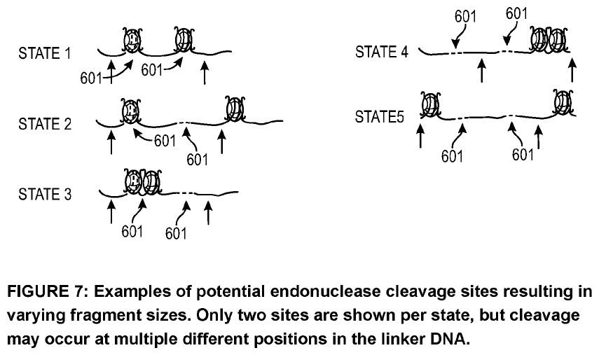

Based on the nucleosome configurations observed using ATM, the smaller ctDNA fragments observed in the data at a lower frequency (between 260 bp and 334 bp) may represent cleavage on one side of a nucleosome and further endonuclease digestion of intervening linker DNA at different positions up to an adjacent nucleosome (FIG. 7; State 2 or 5). Larger fragments (between 334 bp and 440 bp) may result from endonucleases acting on the linker DNA outside of two adjacent mononucleosomes (FIG. 7; States 1, 3 or 4).

Dinucleosome formations may be linked to promoter regions based on their association with RSC, as this complex is enriched at highly expressed genes. Data using a yeast model also demonstrated that all but one of the nucleosomes were removed upon promoter activation, which fits with the model of nucleosome sliding by RSC, unravelling of DNA and ejection of histone octomers along the path, leaving only the single nucleosome bound to RSC at the end of the process. Additional data, using ChIP-Seq in a yeast model where RSC levels are depleted showed an increase in histones upstream and downstream of the transcriptional start site (TSS). Together, these data implicate RSC-mediated repositioning in the removal of nucleosomes in the NDR (nucleosome depleted regions) and regulation of transcription.

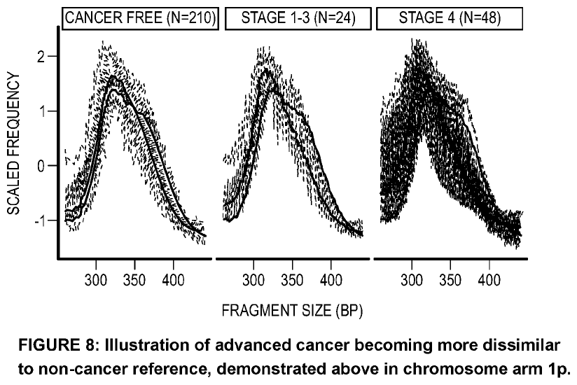

In FIG. 8, the dinucleosomal fragment size densities in chromosome arm 1p for each sample is plotted in blue, the median fragment size density for each sample type in black, and the reference in orange. It is evident from FIG. 8 that samples from stage 4 cancer are even less similar to the reference than samples in stages 1-3. In particular, it is evident that the dinucleosomal fragment size density becomes more skewed and the overall fragments become shorter in stage 4 relative to stage 1-3 cancer and stage 1-3 cancer relative to cancer-free.

Table 2 illustrates the ability of a machine learning model to distinguish cancer from cancer-free individuals using this novel genomic feature extracted from cell-free DNA. The AUC of the machine learning model using dinucleosomal coefficients indicates a strong predictor of cancer status. More indicative of utility for early cancer detection is sensitivity at a high specificity. Combining the two features show an AUC and sensitivity that is as good as or better than the best feature. The results of the combined features demonstrate how this novel summary of fragment size density can complement other genomic features to determine cancer status.

TABLE 2
Cross-validated results of cancer detection using divergence from
dinucleosomal reference per arm.

| Feature Set | AUC | Sens. (95% Spec.) | Sens. (98% Spec.) |
|---|---|---|---|
| Short/Total Coverage | 0.7963 | 0.5342 | 0.5205 |
| Dinucleosomal | 0.8493 | 0.6164 | 0.4931 |
| Dinucleosomal, Coverage | 0.8847 | 0.6164 | 0.5890 |

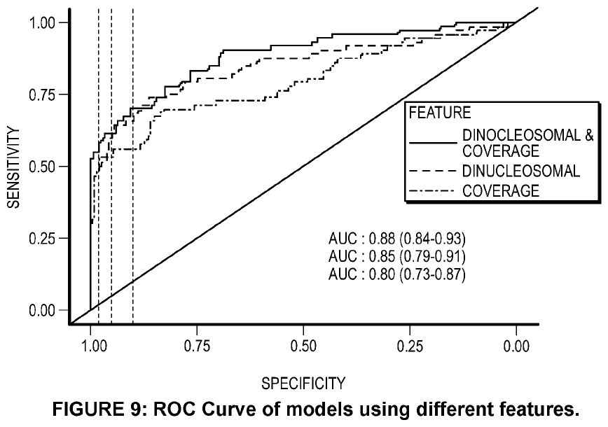

FIG. 9 shows ROC curves of these three models. These curves demonstrate the high sensitivity at even high specificities of the dinucleosomal coefficients as a feature as well the combination of dinucleosomal and coverage features. Reference lines indicate 90%, 95%, and 98% specificity.

Although the invention has been described with reference to the above examples, it will be understood that modifications and variations are encompassed within the spirit and scope of the invention. Accordingly, the invention is limited only by the following claims.

1. A method of determining the cancer status of a subject comprising:
(a) analyzing a shape of a curve of cell free DNA (cfDNA) fragment size density in a sample from a subject, wherein a difference in the shape of the curve of cfDNA from the subject and that from a reference sample of a healthy subject, is indicative of cancer in the subject; and
(b) optionally administering a cancer treatment to the subject.

2. The method of claim 1, wherein analyzing the shape of the curve of cfDNA fragment size density comprises fitting a finite mixture of distributions to counts of fragment sizes.

3-8. (canceled)

9. The method of claim 2, wherein analyzing the shape of the curve of cfDNA fragment size density comprises excluding fragment sizes less than about 10, 50, 100 or 105 bp and greater than about 220, 250, 300, 350 bp or greater.

10. The method of claim 1, wherein analyzing the shape of the curve of cfDNA fragment size density comprises quantifying the shape of the curve using coefficients of a polynomial regression fit to counts of fragments of a given length.

11. (canceled)

12. The method of claim 10, wherein analyzing the shape of the curve of cfDNA fragment size density comprises excluding fragment sizes less than 105 bp and greater than 170 bp.

13. The method of claim 1, wherein analyzing the shape of the curve of cfDNA fragment size density further comprises:
(i) processing a sample from the subject comprising cfDNA fragments into sequencing libraries;
(ii) subjecting the sequencing libraries to low-coverage whole genome sequencing to obtain sequenced fragments;
(iii) mapping the sequenced fragments to a genome to obtain windows of mapped sequences; and
(iv) analyzing the windows of mapped sequences to determine cfDNA fragment lengths.

14-16. (canceled)

17. The method of claim 13, wherein a cfDNA fragmentation profile is determined within each window.

18-26. (canceled)

27. A method of predicting cancer status of a subject comprising:
(a) analyzing a shape of a curve of cell free DNA (cfDNA) fragment size density in a sample obtained from the subject;
(b) comparing the shape of the curve of cfDNA fragment size density of the sample to a reference curve shape; and
(c) detecting cancer in the subject when the shape of the curve of cfDNA fragment size density of the sample is different from the reference curve shape, thereby predicting the cancer status of the subject.

28. The method of claim 27, wherein analyzing the shape of the curve of cfDNA fragment size density comprises fitting a finite mixture of distributions to counts of fragment sizes.

29-34. (canceled)

35. The method of claim 28, wherein analyzing the shape of the curve of cfDNA fragment size density curve comprises excluding fragment sizes less than about 10, 50, 100 or 105 bp and greater than about 220, 250, 300, 350 bp or greater.

36. The method of claim 27, wherein the reference curve shape is a shape of a curve of cfDNA fragment size density measured in a sample obtained from a healthy subject.

37. The method of claim 27, wherein the cancer is selected from the group consisting of: colorectal cancer, lung cancer, breast cancer, gastric cancers, pancreatic cancers, bile duct cancers, and ovarian cancer.

38. The method of claim 27, wherein the step of comparing comprises comparing the shape of a curve of cfDNA fragment size density to a reference curve shape over the whole genome.

39. The method of claim 27, wherein the step of comparing comprises comparing the shape of a curve of cfDNA fragment size density to a reference curve shape over a subgenomic interval.

40-55. (canceled)

56. A system comprising:
(a) a sequencer configured to generate a low-coverage whole genome sequencing data set for a sample; and
(b) a computer system having a non-transitory computer readable medium with instructions to:
(i) process the low-coverage whole genome sequencing data set to produce a curve of fragment size density of the sample;
(ii) fit the curve of fragment size density of the sample to at least two different sets of established statistical parameters to produce at least two suggested curve fits;
(iii) display the at least two suggested curve fits, enabling a user to select at least one of said at least two suggested curve fits for further processing; and
(iv) display a suggested curve fit line corresponding to a selected suggested curve fit of (iii) together with a reference curve fit line, enabling a comparison between the selected suggested curve fit line and the reference curve fit line.

57. The system of claim 56, wherein the computer system further comprises:
an extraction unit operable to select suitable components for curve fitting analysis;
a curve fitting unit operable to perform a finite mixture of normal distributions fitting or perform a polynomial regression fitting by using user-defined equations;
a curve fitting goodness analysis unit operable to provide indicators for fitting quality generated by the curve fitting unit;
a curve fitting parameters characterization unit operable to classify curve fitting parameters as compared to reference values; and
a characterization database for storing fitting coefficients and their characterization.

58. The system of claim 56, wherein the curve of fragment size density is a curve of cell free DNA (cfDNA) fragment size density of a sample.

59. The system of claim 56, wherein fitting the curve of fragment size density comprises quantifying the shape of the curve.

60. The system of claim 56, further comprising a visual display device operable to display the curve fit line, the reference curve fit line, and a comparison of both.

61. The system of claim 56, further comprising a printer coupled to the computer system to print a report displaying the curve fit line, the reference curve fit line, and a comparison of both.

62-108. (canceled)
